(19) United States
(12) Patent Application Publication
(10) Pub. No.: US 2005/0124017 A1
(43) Pub. Date: Jun. 9, 2005

(54) Quantitative alkaline-phosphatase precipitation reagent and methods for visualization of protein microarrays

(76) Inventor: Stewart Lebrun, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/000,774

(22) Filed: Dec. 1, 2004

Related U.S. Application Data
(60) Provisional application No. 60/527,242, filed on Dec. 5, 2003.

(51) Int. Cl. G01N 33/53; G01N 33/537; G01N 33/543

(52) U.S. Cl. 435/7.92; 534/727

(57) ABSTRACT

A system and method are disclosed for the rapid, reproducible and inexpensive visualization, imaging and digital analysis of molecular interactions between ligands and proteins immobilized on an addressable two-dimensional microarray.

### RELATED APPLICATIONS

This application claims priority under 35 U.S.C. § 119(e) to U.S. Provisional Application No. 60/527,242, filed on Dec. 5, 2003 which claims priority under 35 U.S.C. §120 to U.S. patent application Ser. No. 10/431,686 filed on May 8, 2003, which claims priority under 35 U.S.C. §119(e) to U.S. Provisional Application No. 60/379,326 filed on May 9, 2002.

### BACKGROUND OF THE INVENTION

1. Field of the Invention

In one aspect of the present invention, a method is disclosed for the rapid, reproducible and inexpensive visualization, imaging and digital analysis of molecular interactions between ligands and proteins immobilized on an addressable two-dimensional microarray.

2. Description of the Related Art

Various conventional approaches have been used to visualize the surface of biological samples, e.g., DNA spots of a microarray such as a DNA chip, protein bands in a one dimensional (1-D) or two dimensional (2-D) gel, etc. For example, a DNA chip is generally a rigid flat surface, typically glass or silicon, that may have short chains of related nucleic acids spotted, e.g., DNA spots, in rows and columns, i.e., an array, thereon. Hybridization between a fluorescently-labeled DNA and specific locations on the chip can be detected and analyzed by computer-based instrumentation. The information derived from the results of hybridization to DNA chips is stimulating advances in drug development, gene discovery, gene therapy, gene expression, genetic counseling, and plant biotechnology.

Among the technologies for creating protein and/or nucleic acid chips are photolithography, “on-chip” synthesis, piezoelectric printing, and direct printing. Chip dimensions, the number of deposition sites (sometimes termed “addresses”) per chip, and the width of the spot per “address” are dependent upon the technologies employed for deposition. The most commonly used technologies produce DNA spots with diameters of 50-300 μm. Photolithography produces spots that can have diameters as small as, for example, 1 micron. Technologies for making such chips are known to those skilled in the art and are described, for instance, in U.S. Pat. Nos. 5,925,525, 5,919,523, 5,837,832, and 5,744,305; which are all incorporated herein in their entirety by reference.

Hybridization to DNA chips can be monitored by fluorescence optics, by radioisotope detection, and by mass spectrometry. There are two main methods conventionally used for the detection of hybridization on planar microarrays. Both employ a fluorescently-labeled DNA, a computerized system, a movable microscope stage, and DNA detection software. Differences occur within the computerized system, which features either a confocal fluorescence microscope (or an epifluorescence microscope) or a charge-coupled device (CCD) camera. Technical characteristics of the microscope system is described in U.S. Pat. Nos. 5,293,563, 5,459,325, and 5,552,928; which are all incorporated herein by reference. Further descriptions of imaging fluorescently immobilized biomolecules and analysis of the images are set forth in U.S. Pat. Nos. 5,874,219, 5,871,628, 5,834,758, 5,631,734, 5,578,832, 5,552,322, and 5,556,529; which are all incorporated herein by reference.

Fluorescence (or epifluorescence) microscopes generally have sets of optical filters that allow for viewing of fluorescent images. For example, the DNA that is hybridized to the surface of the DNA chip is typically labeled with fluorescent molecules that absorb light at one wavelength and then emit a different wavelength. The microscope may be equipped with sets of optical filters that block the wavelengths of light from the light source associated with the microscope but which allow the light emitted by the fluorescent molecules to pass therethrough such that the light may reach the eyepiece or camera. The light source is typically integral with the microscope and is an important part of the imaging system.

These conventional microscopes are sophisticated and expensive instruments that require training and maintenance. A single microscope objective typically has multiple lenses that are generally very expensive. A lens generally refers to a transparent solid material shaped to magnify, reduce, or redirect light rays, e.g., focus light. A light filter or mirror is distinct from a lens. Furthermore, use of a microscope requires a dedicated workspace that is approximately the size of a typical desk. Conventional microscopes have a light path that is several centimeters long that transmits collected light through air and other assorted optical devices within the light path. One of the challenges in microscopy is making the microscope as efficient as possible in capturing all of the light that leaves the sample surface so that an optimal image can be captured.

A CCD is a silicon chip, whose surface is divided into light-sensitive pixels. When a photon hits a pixel, it registers a tiny electric charge that can be counted. Therefore, with large pixel arrays and high sensitivity, CCDs can create high-resolution images under a variety of light conditions. A CCD camera incorporates a CCD to take such pictures. Included with the camera is an arc lamp with different filters to produce different excitation wavelengths. The camera then collects the emitted fluorescent light, resulting in the desired image.

CCDs offer increased sensitivity and resolution. This enables the capture and production of precise intensity measurements of very faint and bright signals in a single image. Unfortunately, CCDs consume relatively large amounts of power, usually work over a smaller area, and are limited in their multi-color capabilities.

The costly instrumentation conventionally used to image biological samples, e.g., protein and DNA chips, impedes the broad usage of such technologies. Therefore, an inexpensive, low-maintenance alternative spot detection method and apparatus for biological sample analysis, e.g., protein and DNA chip analysis, that is easy to use and requires a minimum of space and maintenance is needed.

### SUMMARY OF THE INVENTION

In a preferred embodiment, the present invention is related to a new quantitative alkaline-phosphatase precipitation reagent and procedure.

In one embodiment, the present invention is related to a rapid and economical method for visualization of microarrays. In a preferred mode, the method is adapted for analysis of protein arrays. Briefly, proteins are spotted on a suitable surface in an addressed format with an opaque background, preferably a solid white background. The protein, DNA, or antibody array is incubated with molecules of interest (antibodies, serum, proteins, drugs or other molecules) washed and then incubated with a detector (secondary antibody labeled with alkaline phosphatase or biotin) or other suitable detection system that can produce a color change at reactive sites. The detector is then visualized using an alkaline phosphatase (an enzyme isolated from calf intestines) catalyzed biotin/streptavidin precipitation reaction. The precipitation reaction results in a sharp color that appears only where AP has been immobilized. The reaction rates for this enzyme remain linear over time, and sensitivity can therefore be improved by allowing the reaction to proceed for longer periods of time.

In another embodiment, visualization of the aforementioned method is enhanced by colorimetry (or, a type of method used to measure color and to define the results of the measurements). The color is digitally captured using a scanning apparatus in conjunction with novel software. This allows for a lumens analysis of the color density, which directly correlates to interactions between immobilized biological samples and various test substances. This data can then be quantified and corrected-using a standard curve and calibration markers, so as to convert the color data to molecular data.

More particularly, a preferred embodiment of the present invention relates to a method for visualization and digital analysis of sample interactions with a biological array. The method comprises the steps of (1) allowing the sample to interact with the array, which comprises biological molecules immobilized on a solid substrate in a two-dimensional and addressable pattern; (2) contacting the array with a secondary detector molecule comprising an enzyme; (3) incubating the array with a developing agent comprising a substrate of the enzyme, such that the enzyme catalyzes a reaction wherein the substrate is converted to a detectable product; (4) digitizing an array image created by the detectable product by scanning the array on a digital scanner; and (5) analyzing the digital image.

Preferably, the detectable product is selected from the group consisting of a colorometric precipitate, a colorometric enzyme-analyte, a colorometric dye-analyte, a colorometric intermediate-analyte, and a radioactive-analyte.

In one mode, the array is washed to remove unreacted sample prior to contacting the array with the secondary detector molecule. In addition, or in the alternative, the array may be washed prior to digitizing the array image in order to terminate the reaction. Preferably, the array is dried prior to digitizing the array image.

In one preferred embodiment of the present method, the enzyme is Alkaline Phosphatase. Likewise the substrate is preferably BCIP/NBT.

In preferred embodiments, the scanner is selected from the group consisting of Epson PERFECTION 1650, Canon CANOSCAN N1240U, and Hewlett-Packard SCANJET 5300C.

In an alternative mode, the array is placed together with one or more additional arrays in a template having from about 1 to 20 array slots prior to scanning the array.

In another alternative mode, the array is labeled with a barcode.

In a preferred mode of the invention, the method further comprises a step of correcting for user error and slide variations using an imaging program. The method may also comprise quantifying markers to construct a standard curve, such that visual intensity can be converted into molecular mass. A clinical index may be calculated by dividing the molecular mass by the dilution factor.

In another embodiment of the present invention, a method is disclosed for the analysis of sample interactions with a protein microarray. The method comprises (1) allowing the sample to interact with the microarray, comprising a plurality of proteins immobilized on a solid substrate in a two-dimensional and addressable pattern, the solid substrate comprising a barcode for sample identification and a PVDF membrane adhered to a rigid support; (2) contacting the microarray with a secondary detector molecule comprising a selective binding moiety and an enzyme conjugated thereto; (3) washing the microarray to remove unbound secondary detector molecules; (4) incubating the array with a developing agent comprising a substrate of the enzyme, such that the enzyme catalyzes a reaction wherein the substrate is converted to a detectable product; (5) washing the microarray to terminate the reaction and remove unreacted developing agent; (6) scanning the microarray using a digital scanner to create a digital image of the microarray; and (7) analyzing the digital image which corresponds to the sample interactions with the protein microarray.

In another embodiment, the present invention relates to a system for analysis of autoimmune diseases in humans, comprising: a microarray, comprising a plurality of autoimmune markers immobilized on a solid substrate in a two-dimensional and addressable pattern, the solid substrate comprising a PVDF membrane adhered to a rigid support; a first reagent comprising anti-human IgG conjugated to an enzyme; a second reagent comprising a developing agent comprising a substrate of the enzyme, wherein the first and second reagents react when combined to yield a colorometric change; and a flatbed digital scanner.

In another embodiment, a system is disclosed analyzing sample interactions with a biological array. The system comprises an array, comprising a plurality of biological molecules immobilized on substrate in a two-dimensional and addressable pattern, the substrate comprising a rigid support which has an opaque surface or has been modified to have an opaque surface, wherein the surface is adapted to bind the plurality of biological molecules; a flatbed scanner adapted to produce a digital image of the array; and a software program adapted to quantify and analyze the digital image.

### BRIEF DESCRIPTION OF THE DRAWINGS

FIG. 1 shows (a) an example of a barcode on the microarray chip with the equivalent numerical value on the left-hand side, along with the chip type and company name; and (b) a blank chip with a barcode and a label (left). The total capacity for the printing area is 30,000 spots. A microarray chip with designated areas (circles) for 10 samples (right). Each circle allows maximum 900 spots and the total capacity of the chip is 9,000 features.

FIG. 2 shows a template secured on the scanner surface.

FIG. 3 shows the scanned microarray image exhibiting spots reactive to the serum from the patients. The intensities of the spots reflect the degree of reactivity.

FIG. 4 shows (a) the SPOTWARE software interface, previewing the microarray to be analyzed. Images are scanned with a false-color, 24-bite color setting at 1600-dpi. FIG. 4(b) shows an expanded view of a portion of the microarray chip, isolating the area within which proteins have been spotted. FIG. 4(c) shows an expanded view of a portion of the microarray, isolating select spots.

FIG. 5 shows an image of the PHOTOSHOP program used to determine the mean value of the spot's luminosity.

FIG. 6 shows the gridding isolates individual spots so that actual intensities for each spot can be extracted.

FIG. 7 shows the interface of the IMAGETOOL software.

FIG. 8 shows a schematic representation of a typical quantification series. As the amount of measured protein increases, so does the lumen value.

FIG. 9 shows an IgE calibration curve.

FIG. 10 shows quantificatioin of IgE binding to allergen, OVA.

FIG. 11 shows a schematic representation of the immunochemistry applications used with the microarray. Chemistry used to detect (a) protein-antibody interactions, (b) antibody-protein interactions, and (c) protein-protein interactions.

FIG. 12 shows an autoimmune disease diagnostic panel. 12 Antigens in various concentrations have been printed onto immobilized PVDF in buffer described above. Lupus patients show a distinct response although not exactly the same to this set of disease markers. For reference each sub-array is 0.5 uM.

FIG. 13 shows substrates detecting SLE diseased markers at various titers with corresponding control titer substrates. As expected, the substrate becomes more sensitive to background as the titer increases.

FIG. 14 shows (a) false color results of the SLE 1:100 dilution of the SLE patient/control patient antibody, with the corresponding list of positive antigens; and (b) quantified results of this same dilution.

FIG. 15 shows (a) false color image of the antibody-protein assay, along with their corresponding protein concentrations; and (b) quantified results of this assay.

FIG. 16 shows (a) the microarray chip with the corresponding quantitative results for the assay developed with RA control patient pool; and (b) the microarray chip with the corresponding quantitative results for the assay developed with the RA patient pool.

### DETAILED DESCRIPTION OF THE PREFERRED EMBODIMENT

In a preferred embodiment, the present invention provides inexpensive methods for resolving colorimetric density representative of interactions between immobilized biological samples (e.g., protein or nucleic acid spots on a microarray) and various test substances. As used herein, biological samples refers to biological material (proteins, nucleic acids, tissues, etc.) associated with a biological material holding structure (e.g., a microarray substrate such as a protein or DNA chip substrate, a gel, etc.) in a manner that allows for detection of the biological material, or portions thereof (e.g., with the use of markers such as dyes, tags, labels, or stains), such as through the use of imaging (e.g., direct mapping).

One or more embodiments of the present invention are operable for use in multiple imaging applications, e.g., imaging of two-dimensional and three-dimensional objects, such as fluorescence imaging, reflective imaging, bar code imaging, densitometry, gel documentation, or in any other application wherein imaging of a biological sample is beneficial. One or more of the systems and methods as described herein may be used for ultra-sensitive sample detection. One or more of the imaging systems and methods of the present invention are flexible (e.g., can image various objects and perform various types of imaging such as fluorescence and reflective imaging) light imaging systems with the ability to produce high-quality images from, for example, various biological sample configurations that use, for example, single color fluorescence, multiple color fluorescence, chemi-luminescence, chemi-fluorescence, colorimetric detection, densitometry, or any other technique detectable through imaging. Such image quality, e.g., spatial resolution, is dependant, at least in part, on the lens and electronic light detector used in such systems. Such imaging provides the ability for filmless detection.

Portions of the following description are primarily provided, for simplicity, with reference to use of microarrays such as protein chips. However, one skilled in the art will recognize that the present invention is applicable to any imageable biological sample, e.g., DNA chips, 1-D gels, 2-D gels, blots, substrates having biological material thereon. For example, as previously noted, such systems and/or methods may be used to image two-dimensional gels, e.g., labeled protein bands of such gels. Thus, polypeptides separated according to the independent parameters of isoelectric point and molecular weight (e.g., protein bands) can be imaged using the present invention.

An imaging system according to the present invention may be used to replace expensive optical detection systems currently employed for microarray analysis. In general, one embodiment of such a system may include an electronic light detector array, a filter, and, optionally, a mapping lens apparatus that enables a microarray to be mapped onto the electronic light detector array. For example, each position on the microarray surface has a corresponding position or set of positions, i.e., detector pixels, on the electronic light detector array. Light associated with the biological material at an address on the microarray surface is received or sensed at one or more known addressed detector pixels or set of detector pixels. Such detector systems are disclosed in U.S. Pat. Appl. No. US 2002/0018199 A1, which is hereby incorporated in its entirety by reference thereto.

In a preferred embodiment of the present invention, a reacted microarray, developed using a variety of applicable detection chemistries (e.g., labeled antibodies, enzyme-linked assays), may be analyzed by scanning the microarray using a linear (rather than a two-dimensional) array of detectors, e.g., in a conventional digital (usually flatbed) scanner. Preferably, the microarray substrate is opaque, thereby facilitating imaging using a conventional flatbed scanner. More preferably, the microarray substrate is white, so the background is minimized. The conventional flatbed scanners are inexpensive and readily available. Their use eliminates the need for a complicated microscope that requires maintenance and trained personnel. By eliminating many lenses, the disadvantages stemming from use of many lenses are reduced.

### Microarray Imaging

Acquisition

The protein microarray is incubated with a sample (e.g., human serum, proteins, antibodies, drugs and other ligands) expected to interact with the immobilized polypeptides. The array is washed and then incubated with a secondary detector molecule. The detector molecule in this example is conjugated with Alkaline Phosphatase (AP). The array is then incubated with an enzyme substrate, such as BCIP/NBT substrate. BCIPINBT (blue-violet) is one of the most sensitive enzymatic substrates because of the significant increase in reaction product with longer incubation time. Another advantage of the BCIP/NBT substrate is that it can be dehydrated and cleared from the array after processing.

The array is washed and the precipitation reaction stopped as a result of washing away the required reagents. The array is then air dried in a dust free environment and transferred to a flatbed scanner, which includes a pre-fitted template. The following scanners have been used with the following protocol and resolutions: Epson PERFECTION 1650, Canon CANOSCAN N1240U, Hewlett Packard SCANJET 5300C, and most recently, the Epson PERFECTION 2400 PHOTO. Any scanner can be used in accordance with the preferred embodiments of the present invention to image the dried microarrays.

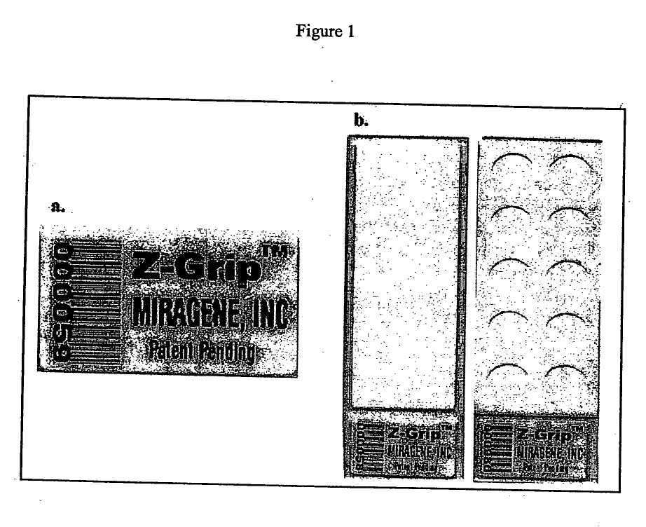

In accordance with one mode of the present invention, preprinted labels (FIG. 1a) with barcodes of specific numerical sequences are included on the microarray chips and/or chip templates. The barcodes may be read by a handheld scanner, or by the imaging software to expedite the data processing by relating each chip with the types of protein, antibodies, patient information and the treatments stored in a database. Based on particular type of chips, the barcodes can be divided into five or less segments corresponding to the different information. Barcodes can be used as an ID for the specific chip. They may be etched on the chip, printed on an adhesive label and applied to the chip. In addition, a duplicate barcode ID from a patient sample, may be transferred to the chip to identify the patient sample. The barcodes may also serve as a landmark for the scanning equipment and software to facilitate addressing of individual spots on the array.

Labels are attached to the array at different times. At first, the company name and types of the chip are printed on the blank labels. These labels are also punctured with holes for the sample depositions with diameters from 2 to 9 mm (FIG. 1b). The number of areas for the sample deposition varies from 1 to 20 depending on the types of analyses used. Then, the labels are attached to the blank chips. The barcodes related to the antibodies are added to the chip prior to the microarray printing. Similarly, the barcodes with patient and treatment information may be applied to the chip whenever the information becomes available.

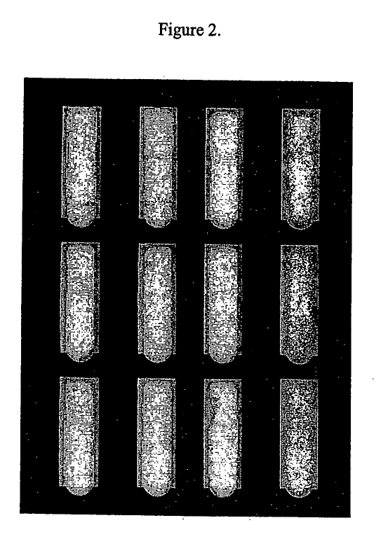

The template may be made from a relatively soft but durable material, such as plastics, with openings (1 to 20) to hold the chips. The template may be secured on a scanner surface so that the relative position remains constant during scanning (FIG. 2).

The microarray chips may be placed into the openings of the template and secured on a flat-bed scanner. In a preferred embodiment, the chips may be secured with suction cups or hands. Gloves should be used to avoid direct contact of the skin with samples.

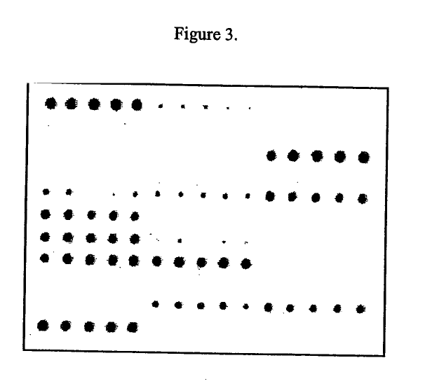

The scanner mode is preferably set to a high resolution, preferably about 1600 dpi. The choice of the scanning resolutions depend upon the needs. Lower resolutions offer faster scanning, smaller image file sizes, but lower image qualities. Workable settings are 600-dpi, 800-dpi, 1200-dpi, 1600-dpi and 2400-dpi. It is preferred to observe the whole scanning area by using the previewing mode prior to scanning. Select and zoom into specific areas of interest containing the desired microarray spots can be selected and magnified using conventional zoom settings. Once the areas of interest are visible in the previewing mode, the microarray can be scanned and the images can be saved on a directory for subsequent visualization and analysis (FIG. 3).

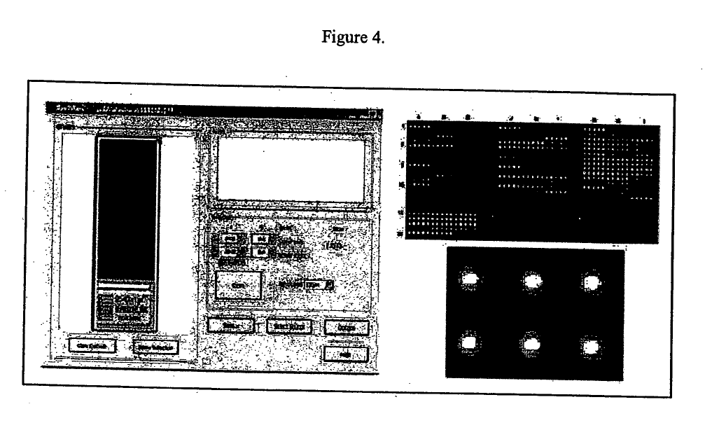

Another option is to use the SPOTWARE Software (Telechem, Sunnyvale, Calif.; a software package designed specifically to acquire microarray images) in conjunction with the flatbed scanner. This software allows for direct capturing of the microarray, without the hassle of previewing the whole scanner area and then zooming in to scan the whole chip. Instead, it is possible to preview just the chip, and to zoom into a particular area of the chip. FIG. 4a illustrates the interface of the software. Settings include a choice of ‘16-bite grayscale’ or ‘24-bite color’, and ‘invert light to dark’ or ‘view as false color.’ Typical settings use 24-bite color viewed as false color at 1600-dpi (where dpi is set on the scanner). FIG. 4a previews the microarray chip with these settings. The false color distinguishes positive signals very clearly, making it easier on the eye and to analyze. Once the chip is previewed, specific portions of the microarray can be viewed and saved. FIG. 4b illustrates a zoomed portion of the chip, showing the area of all the spotted proteins. FIG. 4c is a further zoomed portion of the chip, isolating only a select few positive protein spots. The SPOTWARE program gives a signal to noise ratio of 16,000 to 1, and a resolution of 10-μm. From here, images can be saved on a directory for subsequent visualization and analysis.

Analysis

Image analysis software is preferably used to analyze the microarray data. In general, a scanned image is opened and the average intensity of each spot is determined with the background contributions eliminated. There are a number of software packages that can accomplish this, including Adobe PHOTOSHOP (6.0 or higher), ARRAYVISION, and IMAGETOOL.

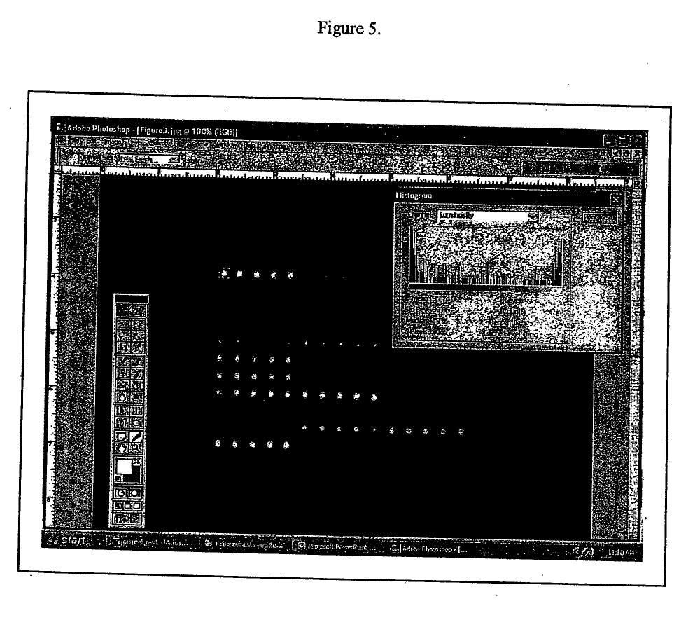

When opening the scanned images in PHOTOSHOP, typically the first step is to adjust the autolevels of the microarray chip. Then, depending upon whether or not the image was acquired via the flatbed scanner, the color may need to be inverted, to give a black background and light spots. This step is not necessary when using SPOTWARE, as images can be given in false color. If desired, the image may be zoomed into, to get a clearer image of the spots, and to aid in the next step. Then, using the rectangular marquee tool, individual spots are highlighted, and the histogram observed. The mean value of the luminosity is then recorded. FIG. 5 illustrates how the mean luminosity is obtained from an inverted image acquired with only the flatbed scanner.

The marquee can then be dragged over the next positive spot, and the luminosity for this, recorded. For PHOTOSHOP, the same marquee is preferably just dragged over the spot of interest, thereby keeping the amount of pixels being observed consistent. The marquee is preferably also dragged over the background so that spot values can be normalized against this. Typically, the background value is close to, if not equal to, 0. Once the luminosity of the series of spots has been recorded (each protein is preferably spotted in replicates, e.g., 2-10 times; the data in the Figures show replicates of five), the average value is taken, and the background, subtracted. This gives a single intensity value for each spotted protein.

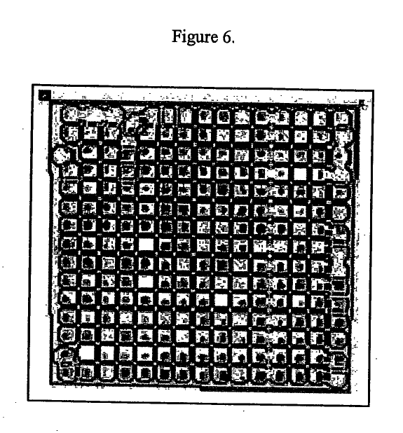

For ARRAYVISION, the steps of analyses include addressing or gridding the spots (FIG. 6), segmentation to distinguish the foreground from the background, as well as the intensity extraction and data storage. Suitable software are developed for the image analyses. 100611 The extracted intensity of the spots are analyzed by querying the database. The spots related to the targets are selected and their intensities may be compared with the threshold values. When the intensities are found to be above the thresholds, the software raises a flag or a warning to inform the user about a possible positive sample. Note that in FIG. 6, the image analyzed was acquired directly from the flat bed scanner. It is also possible to first invert the colors in PHOTOSHOP and then open the image in ARRAYVISION, or to use the false color image scanned via SPOTWARE.

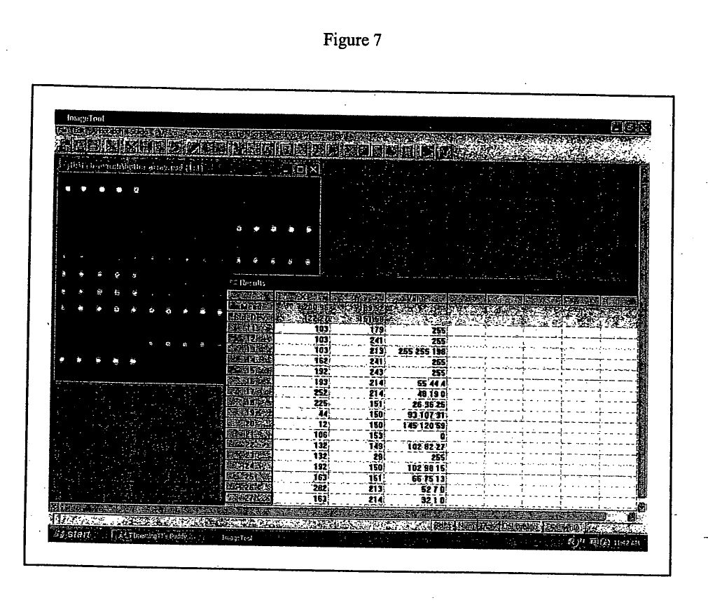

IMAGETOOL has many advantages over the other two analysis software packages. Once in the program, the user simply needs to open the image, select the analyze points option, and click on points within the microarray chip. The program will automatically record both the location of the selected point on the chip, along with three values of the intensity within the selected point as seen in FIG. 7.

Another advantage of this program is that, in conjunction with the flatbed scanner, it can acquire the image directly from the scanner. IMAGETOOL will go directly to the scanner program so that the image can be scanned as normal. Once scanned, the image automatically opens in IMAGETOOL to be analyzed.

Quantification and Correction

Regardless of which software is used, a first step to quantification in accordance with a preferred embodiment of the disclosed method is to input all lumens values into a spread sheet, such as Microsoft EXCEL, and if necessary, average these values to one number per spot. In general, quantification occurs by first determining the average intensity value for each protein, along with its standard deviation can be determined. These intensity values can be converted into mass values, thus quantifying protein hybridization.

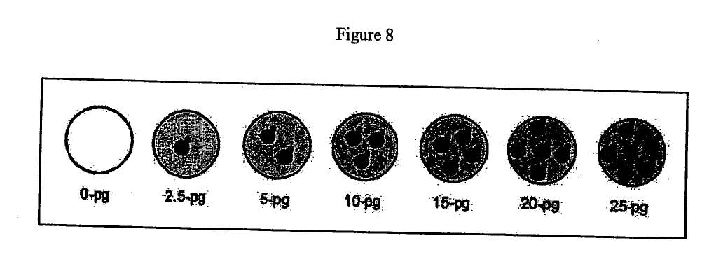

More specifically, each analyzed chip has a quantification series, where the quantification series is the known mass of the measured protein. Typically, the series uses known proteins ranging from mass 0-pg to 25-pg. FIG. 8 is a schematic representation of a quantification series. As the amount of measured protein increases, so does the lumen value.

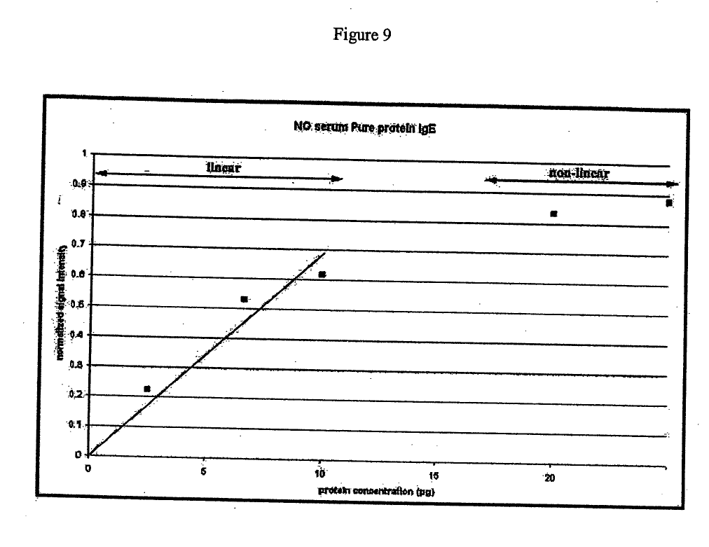

For example, the IgE antibody binds in a 1:1 ratio with the OVA allergen. Then, a calibration curve is first created for IgE by plotting the average intensity as a function of the known mass, as seen in FIG. 9.

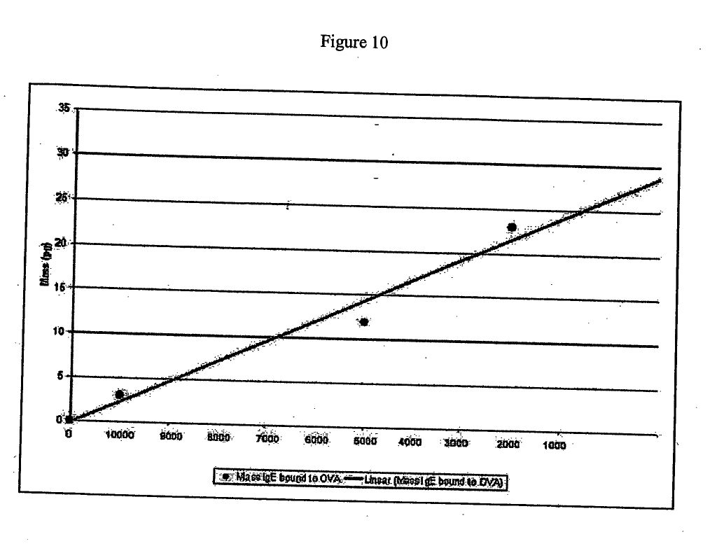

Once a calibration curve has been created, the IgE binding to OVA can be quantified. After analyzing the data for a dilution of OVA (ranging from a 1:10,000 to 1:1,000 titer), the lumens values are converted into mass values. These values are obtained by utilizing the calibration curve shown in FIG. 9, as it gives a relation between the signal intensities and protein mass. Then, the mass of IgE bound to OVA as a function of dilution can be plotted, as seen in FIG. 10.

### Microarrays

An array is used in the present disclosure to mean an arrangement of molecules, particularly biological macromolecules (such as antigens, polypeptides or nucleic acids) in addressable locations on a substrate. A “microarray” is an array that is miniaturized so as to require microscopic examination for evaluation.

In preferred embodiments, the antigens are attached to solid supports. These supports may be plates (glass or plastics) or membranes made of nitrocellulose, nylon, or polyvinylidene difluoride (PVDF), or other suitable material. To facilitate use of conventional flatbed scanners in accordance with a preferred aspect of the present invention, the surface of the solid support may be modified to be opaque, and more preferably, white, in order to minimize the background. In a preferred embodiment, as discussed above, the solid supports are PVDF-coated supports as detailed in co-pending U.S. patent application Ser. No. 10/376,351; incorporated herein in its entirety by reference thereto. Membranes are easier to handle and antigens can be readily immobilized on them. Glass or plastic plates provide rigid support and are therefore necessary in some special applications. Antigens may be immobilized on the solid support directly or indirectly. When interrogated with a sample, the binding of antibodies in the sample to the array (possibly producing a pattern) indicates the relative binding affinity of the antibodies for each of the immobilized polypeptides. Characteristics of binding interactions are discussed in greater detail below.

The term “immobilize,” and its derivatives, as used herein refers to the attachment of a bioactive species directly to a support member or to a support member through at least one intermediate component. As used herein, the term “attach” and its derivatives refer to adsorption, such as, physisorption or chemisorption, ligand/receptor interaction, covalent bonding, hydrogen bonding, or ionic bonding of a polymeric substance or a bioactive species to a support member.

Related methods of immobilizing bioactive molecules, in particular, nucleic acids, on polymeric substrates are disclosed in U.S. Pat. No. 5,897,955 to Drumheller and U.S. Pat. No. 6,037,124 to Matson; the disclosures of which are incorporated herein in their entirety by reference thereto.

This work resulted from attempts to perform immunochemistry, using antigens printed by a commercial DNA/RNA/Protein printer. We found that commercially available substrates and chemistries developed for nucleotides are not optimal for antigen binding or immunochemsitries. Various derivitized slides including aldehyde, epoxide, amine, L-Lysine where not adequate for our requirements. Our suspicion is that binding chemistries utilized to linerize nucleotides for hybridization are not optimal for protein-protein or protein-antibody interactions. It is likely that aggressive binding of these substrates destroys secondary and tertiary protein structures and to the extent these structures are altered, epitopes vital for immuno or protein-protein assays are altered.

PVDF membrane is often used for the western blotting technique. This method involves a pre-soaking step of membrane in methanol to solubilize and the addition of methanol to buffers. The membrane must be kept in the methanol buffer or proteins will not transfer to membrane. This is often the case when there are large areas on a membrane where there was no transfer due to a bubble. In addition to being hydrophobic, PVDF membrane is hard to handle and will not lye flat during printing. These physical and chemical limitations make PVDF an inappropriate surface for arrays.

We have developed a method to utilize PVDF membrane, sheets or pellets for immunochemistry and protein-protein interaction studies. Two modifications which facilitate use of PVDF membranes are: (1) adhering the PVDF membrane to solid support using silicone, glues, double sided tape or direct chemical bonding to silanated slides, and (2) a printing buffer that both protects protein three-dimensional integrity and allows adherence to PVDF under DRY printing conditions without membrane soaking in methanol and associated diffusion etc. The following provide specific methodological examples and materials which exemplify preferred embodiments of the present invention. Other known methods and materials used for visualization of support-bound molecular species are also encompassed within the present disclosure.

Materials:

Protein-immobilizing polymer: commercially available polyvinylidene fluoride (PVDF) sheets or membranes. PVDF pellets may also be used in some modes of the invention.

Solid substrate: glass slides, plastic or other flat surfaced material.

Adhesion material: commercially available silicon sealant, epoxy or other glue, or suitable double-sided tape.

Bonding of Vinyl Fluoride to substrate—a) apply silicon, glue or double sided tape to solid substrate in even thin layer, b) under clean conditions, place sheet on lab bench and apply solid substrate (glue side facing PVDF sheet) to vinyl fluoride sheet, and c) press firmly and allow drying. Using a sharp instrument (e.g., a razor blade, exacto knife, etc.), cut sheet so that it is size of solid substrate.

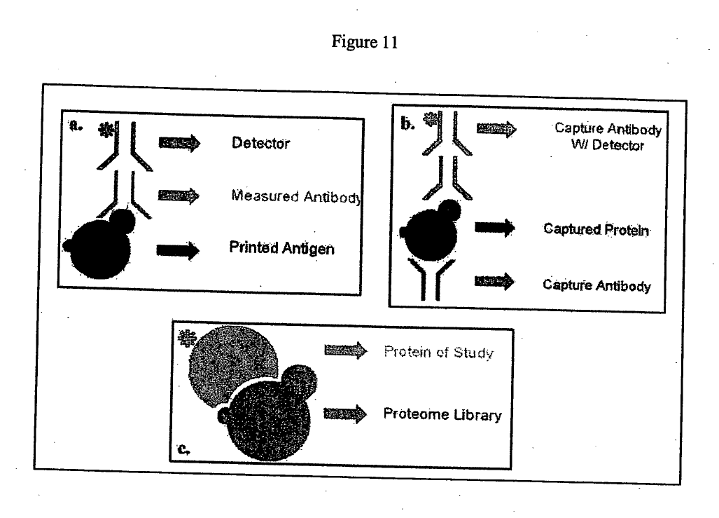

Immunochemistry applications—There are three main types of interactions currently under investigation—protein-antibody (where the system is referred to as the antibody assay), antibody-protein assay (where the system is referred to as the protein assay), and protein-protein interactions. These are shown schematically in FIG. 11.

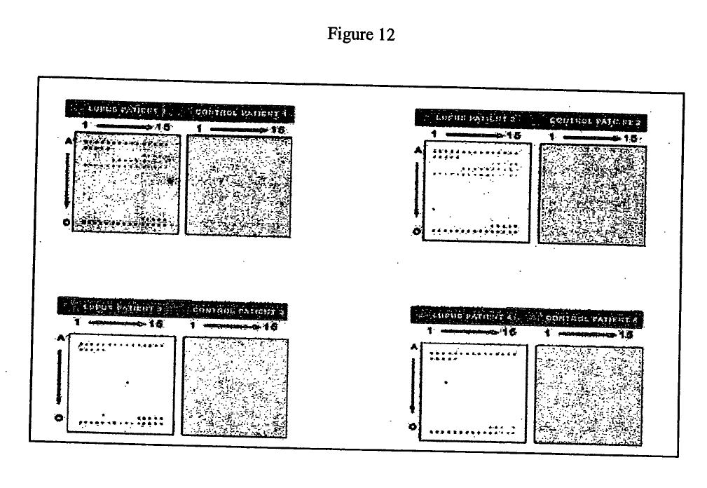

In regards to protein-antibody interactions, specific research has been geared towards analyzing and finding disease markers for certain auto-immune diseases. In our earlier work, the surface was used to determine differences in immunoreactivity to autoimmune disease related markers between 4 Lupus patients and 4 age/sex-matched controls. Antigens were printed in 8 replicate arrays on substrate at a concentration of 1 mg/ml in optimized buffer. The array was blocked with Casein in TBS, followed by patient serum in a titer of 1000 and incubated with arrays for 1-hr. The arrays where then washed 3× in PBS and a secondary anti-human IgG conjugated to Alkaline Phosphatase was added (Pierce Biochemicals, Rockford Ill., Goat anti human IgG Alkaline Phosphatase Conjugated Product #31310) After 1 hr the arrays were washed 3× in PBS and a developing reagent was added (1-step BCIP/NBT, Pierce Biochemicals). After 15 minutes slides were washed, allowed to dry and scanned in a commercial scanner. Results are shown below (FIG. 12). Although Alkaline Phosphatase conjugated secondary antibody was used, this method would is compatible with protein A conjugated Alkaline phosphatase or secondary antibodies labeled with other enzymes (HRP) or dyes (fluorescent etc). FIG. 12 shows the background and specificity of this substrate in this use and utility for immunochemistry applications.

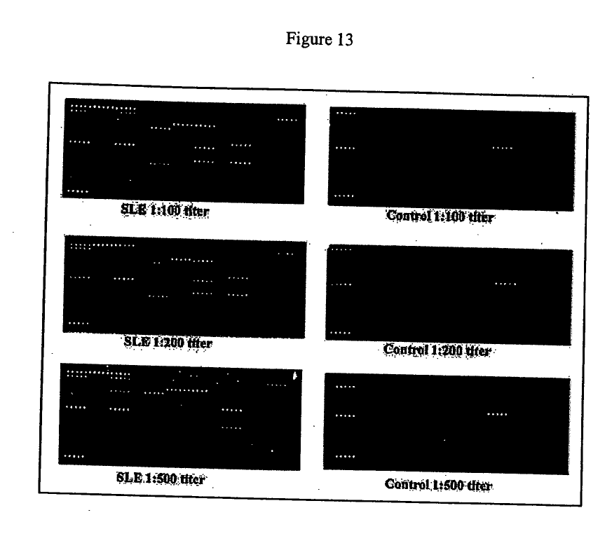

Systemic lupus Erythematosus (SLE) disease marker's were confirmed and quantified. Similarly, a number of antigens (potential SLE disease markers) were printed onto 6 substrates, followed by the 1-hr incubation of the substrate with Casein in TBS. Three different titers (100, 200, and 500) of a pool of 10 SLE patients and three corresponding titers of 10 SLE control patients were used to incubate the substrates for another hour. Following, the substrates were washed three times in 1×-PBS followed by another 1-hr incubation in a 1:10,000 dilution of anti-human IgG conjugated to Alkaline Phosphatase. Again, the substrates were washed three times, and were then developed and washed as above. False color results are shown in FIG. 13.

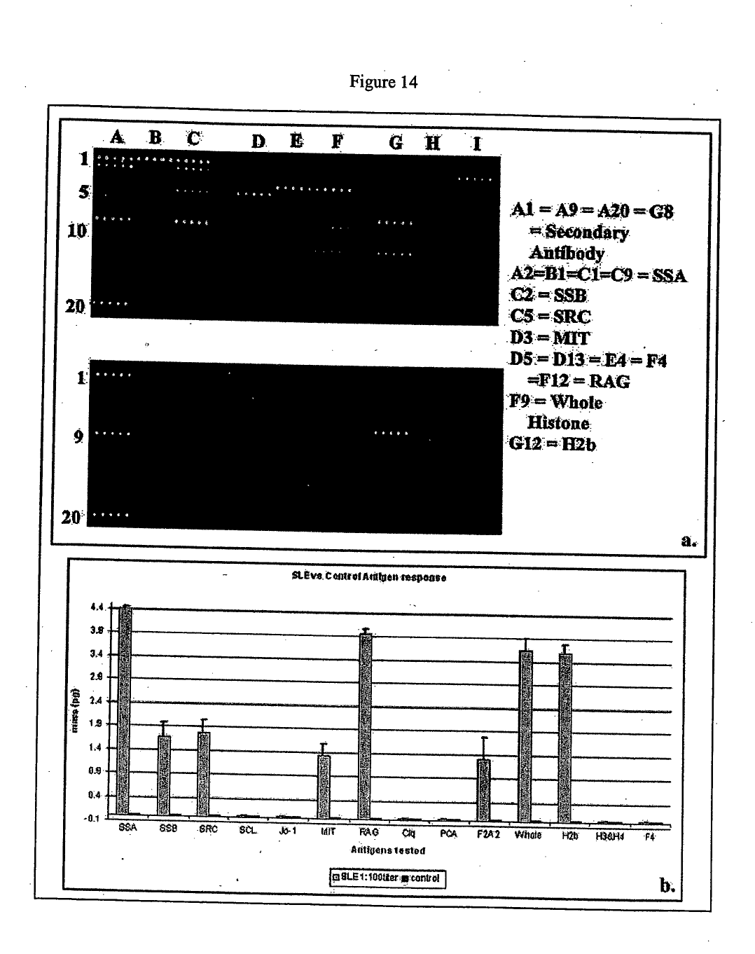

It is noted that the titer signal increases as the antibody titer increases, as does the background noise. FIG. 14a illustrates which markers came out positive using the 100 titer of the SLE patient pool and SLE control patient pool antibodies, where FIG. 14b illustrates the quantified results at this titer.

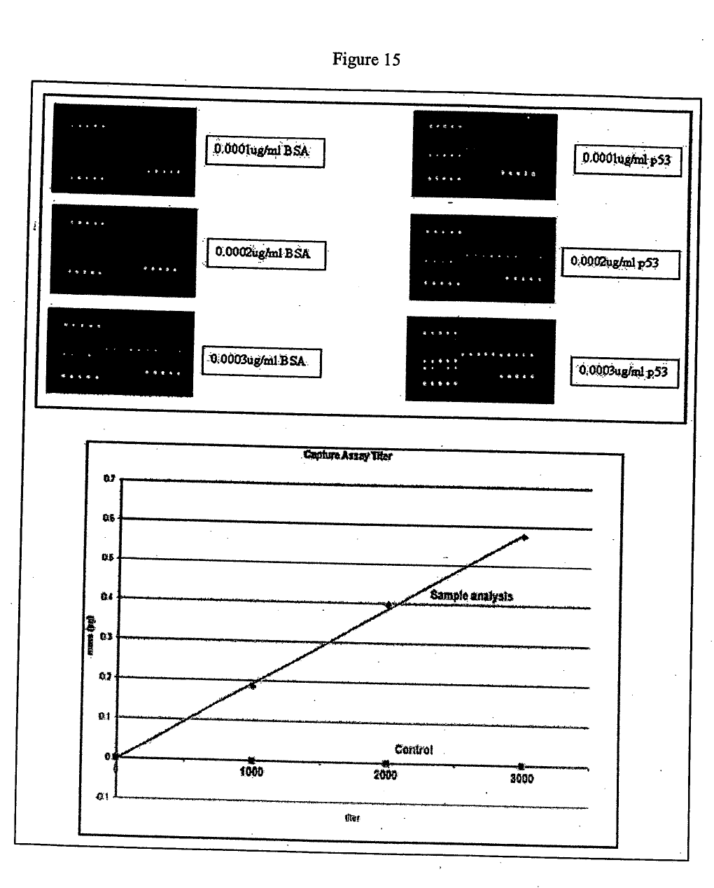

The substrate and analysis technique described above has also proven to be effective in detecting antibody-protein interactions. In one experiment, anti-p53 antibody was spotted onto six of the above-mentioned microarray chips in serial dilutions. The chips were then individually blocked with 1%-Casein in TBS for 1-hr with agitation. The chips were then incubated for 1-hr in three different concentrations ((0.0001-μg/ml, 0.0002-μg/ml, or 0.0003-μg/ml) of p53 protein or BSA protein, where BSA served as the control protein. The substrates were washed three times (10-min each) in 1×-PBS and further incubated for another 1-hr in a 1:250 dilution of rabbit polyclonal IgG (p53 FL393) to 1×-PBS. Again, chips were washed three times (10-min each) in 1×PBS, and then incubated for 1-hr in a 1:1000 dilution of anti-rabbit IgG-AP to 1×-PBS. Following this was another three washes (10-min each) of the substrate in 1×-PBS, and the development of the chips in developing reagent. After 15-min, the chip underwent its final wash. This process yielded the results shown in FIG. 15. FIG. 15a is the false color image of the chips, and FIG. 15b is the quantitative results.

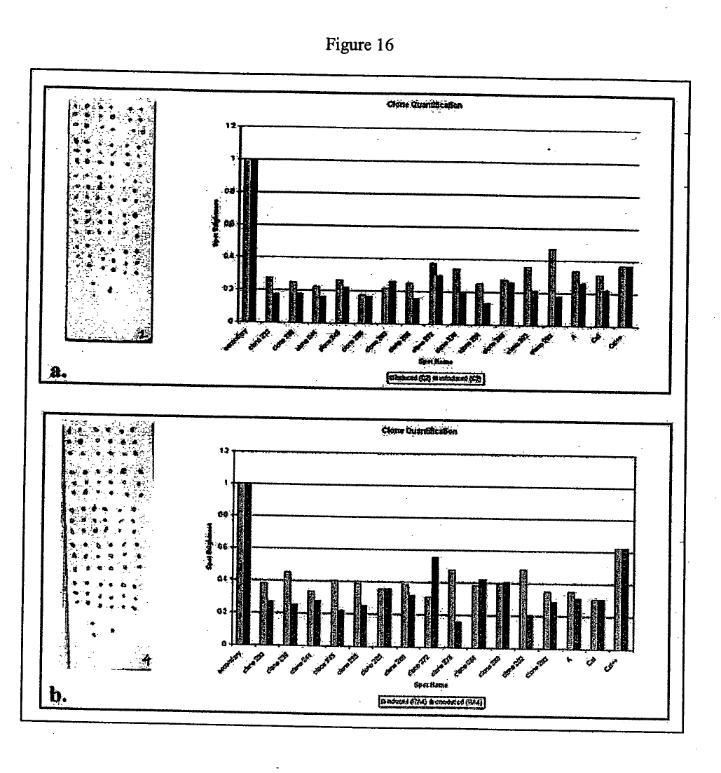

The third interaction currently under study is protein-protein interactions. For this case, a DNA sequence coding for a sutitable marker/tag is first cloned. The DNA sequence is then spliced into a suitable vector containing a cDNA library, where the cDNA library can be excised from the vector utilizing restriction enzyme digestion. The excised cDNA library is then inserted in frame into the vector containing the marker. These cDNA library containing vectors are then used to transfect host cell cultures, where these host cell cultures are carefully selected. The single clone are transferred and amplified, and express the tagged protein. The host cells are then lysed and hand-spotted onto the microarray chip. Following the standard assay protocol, the interaction between the proteome library and desired protein can be detected. More specifically, the substrates are first blocked for 1-hr in 1% Casein in TBS with agitation. Then a dilution of 1:500 RA patient pool (or RA patient control pool) to blocker is used to incubate the substrate for another hour. The substrate is then washed three times (for 10-min each wash) in 1×-PBS, and then incubated for another hour in a 1:1000 dilution of anti-human IgG-AP to 1×-PBS. After washing three times (10-min each) in 1×-PBS, the developing reagent is added. Finally, after 15-min, the final wash is undergone. FIG. 16 illustrates the results of this assay. FIG. 16a is the microarray chip with the corresponding quantitative results for the assay developed with RA control patient pool. FIG. 16b is the microarray chip with the corresponding quantitative results for the assay developed with the RA patient pool.

In a particularly preferred embodiment, a new quantitative alkaline-phosphatase precipitation reagent and methods are disclosed.

Although BCIP/NBT is considered a “sensitive” detector of alkaline phosphate activity, it cannot be quantitative without the introduction of the chromogenic substrate p-nitrophenyl phosphate (Molecular Cloning, A Laboratory Manual, Vol. 3, 3rd Edition. Sambrook, J. and Russell, D. p. A9.41. 2001. Cold Spring Harbor Laboratory Press. New York).

Unexpectedly, the Inventors have found that an alternative to make the BCIP/NBT reaction quantitative, is to avoid introduction of phosphate groups, as detailed below. To develop allergen printed Z-grip chips, they were submerged in Blocker Solution (1% Casein in TBS) and incubated at room temperature for 30 min so as to eliminate unwanted background due to unspecific protein binding. Previous experiments have demonstrated that longer blocking incubation times do not further improve the signal to noise ratio. Following incubation, each chip was treated separately with human serum derived from a patient known to mount an allergic response to three different types of allergen, namely dust mites, grass, and mold. Prior to adding the serum, the stock serum was serially diluted two-fold with commercially available equine serum, which gave a dilution range from 1:1 to 1:256 (“high-positive” to “low-positive”, respectively). Once diluted, the serum was added (100 ul) to the printed section of the chip using a 200 ul capacity micropipette. In order to reduce the amount the amount of sample evaporation that may occur during incubation, each chip was placed in a sealed container. All samples were incubated at room temperature for 2½ hours. Following this incubation period, all treated chips were washed using TBS (Tris Borate Saline) doing three separate washes at 10 minutes each at room temperature. Next, to detect and visualize any potentially bound human IgE antibody/allergen complex, a secondary antibody (anti-human IgE mouse monoclonal antibody conjugated to alkaline phosphatase) was added to each treated chip. In order to be able to detect relatively low amounts of bound human IgE antibody and at the same time reduce unwanted noise, a 1:7.5 dilution of the secondary was prepared with Blocker solution. Previous experiments have demonstrated that using greater dilutions of secondary antibody result in weak to no signal from “low-positive” assays. 100 ul of the diluted secondary antibody was added to each sample chip using a mircropipette. All samples were then incubated at room temperature inside a sealed container as described above. Following this incubation, all samples were washed as described above. A final 10 min wash was performed on all samples using “Alkaline Phosphatase Buffer” (0.1M Tris pH 9.5, 0.1M NaCl, 0.05M MgCl2) so as to equilibrate the Z-grip membrane prior to adding the developer. For preparation of developer solution, Nitro-Blue Tetrazolium Chloride (NBT) was added along with 5-Bromo-4-Chloro-3′-Indolylphosphate p-Toluidine salt (BCIP) at a 2:1 volume ratio, respectively. Upon mixing of the components, the treated chips were submerged in the AP-developer solution and incubated at room temperature while gently rocking for 15 minutes. Upon completion of this incubation, the reaction was stopped with dH2O. The chips were finally air dried and subsequently quantified using specific computer software.

In another embodiment of the present invention, a layer of PVDF may be formed on a solid support by melting the polymer and applying and it to the solid support. Modification of the PVDF chemistry is also deemed to fall within the scope of the present invention. Modifications may include carboxylation, amidization, and introduction of other reactive groups to the PVDF in order to promote immobilization of different bioactive species. In one other embodiment, solid PVDF supports may be prepared by molding of the melted polymer.

Within an array, each arrayed molecule is addressable, in that its location can be reliably and consistently determined within the at least two dimensions of the array surface. Thus, in ordered arrays the location of each antigen, peptide, polypeptide or partially purified lysate fraction is assigned at the time when it is spotted onto the array surface and a key may be provided in order to correlate each location with subsequent antibody binding patterns or fingerprints. Often, ordered arrays are arranged in a symmetrical grid pattern, but antigens could be arranged in other patterns (e.g., in radially distributed lines or ordered clusters). The many spots of an antigen array can be arrayed in the shape of a grid, although other array configurations can be used so long as the spots of the array are addressable.

The shape of the antigen application “spot” is immaterial to the invention. Thus, though the term “spot” refers generally to a localized deposit of antigen or polypeptide, and is not limited to a round or substantially round region. For instance, essentially square regions of polypeptide application can be used with arrays, as can be regions that are essentially rectangular (such as slot blot application), or triangular, oval, or irregular. The shape of the array itself is also immaterial, though it is usually substantially flat and may be rectangular or square in general shape.

In one preferred embodiment of the antigen array, each antigen has been spotted onto the array twice to provide internal controls. Alternatively, a greater number of replicates may be desirable in some instances. Thus, the number of replicates may range from 1 to n, more preferably from 1 to 4 and most preferably from 1 to 2. The duplicate antigens may be positioned in a pair of horizontally adjacent addresses of the array. However, as long as the locations of the duplicate antigens in the array are known, the relative positions are not important.

Arrays may include a plurality of antigens “spotted” at assignable locations on the surface of an array substrate. In certain embodiments, polypeptides are deposited on and bound to the array surface in a substantially native configuration, such that at least a portion of the individual polypeptides within the spot are in a native configuration. Such native configuration polypeptides are capable of binding to or interacting with molecules in solution that are applied to the surface of the array in a manner that approximates natural intra- or intermolecular interactions. Thus, binding of a molecule in solution (for instance, an antibody) to an antigen immobilized on an array will be indicative of the likelihood of such interactions in the natural situation (ie., within a cell). In other embodiments of the antigen array, the peptide/polypeptides may be denatured, reduced and/or otherwise chemically pretreated (e.g., to remove sugars).

In certain arrays of the invention, one or more location/address on the array is occupied by a pooled mixture of more than one substantially pure antigens/polypeptides (e.g., chromatography fractions of a crude cell lysate or tissue extract). All of the locations on the array may contains pools of peptides, or only some of the locations. In some circumstances it may be desirable to array a polypeptide associated with one or more non-target polypeptides, for instance a stabilizing polypeptide or linker molecule. In addition, the native conformation of certain binding sites on proteins can only be assayed for antibody binding when the antigen is associated with other molecules, for instance when a polypeptide natively exists as one subunit of a multimeric complex. Pooled arrays include those on which one or more of the locations contains a multimeric polypeptide complex. In the case of such an array, it is envisioned that different antibody molecules may bind to different determinants within the complex of pooled or linked antigens.

In accordance with one embodiment of the present invention, bound antibody molecules can be stripped from an array, in order to use the same array for another patient sample analysis, once the antibody fingerprint and diagnostic test result are recorded and stored. Any process that will remove essentially all of the bound antibody molecules from the array, without also significantly removing the immobilized antigens of the array, can be used with the current invention. By way of example only, one method for stripping a protein array is by washing it in stripping buffer (e.g., 1 M (NH,)₂SO, and 1 M urea), for instance at room temperature for about 30-60 minutes. Usually, the stripped array will be equilibrated in a low stringency wash buffer prior to incubation with another sample.

As discussed above, antigen arrays in accordance with preferred embodiments of the present invention may use either a macroarray or a microarray format, or a combination thereof. Such arrays can include, for example, at least 50, 100, 150, 200, 500, 1000, or 5000 or more array elements (such as spots). In the case of macro-arrays, no sophisticated equipment is usually required to detect the bound antibody on the array, though quantification may be assisted by known automated scanning and/or quantification techniques and equipment. Thus, macro-array analysis can be carried out in most research laboratories and biotechnology companies, without the need for investment in specialized and expensive reading equipment.

Examples of substrates for arrays include glass (e.g., functionalized glass), Si, Ge, GaAs, GaP, SiO, SiN, modified silicon nitrocellulose, polyvinylidene fluoride, polystyrene, polytetrafluoroethylene, polycarbonate, nylon, fiber, or combinations thereof. Array substrates can 3 be stiff and relatively inflexible (e.g., glass or a supported membrane) or flexible (such as a polymer membrane). One commercially available microarray system that can be used with the arrays of this invention is the FASTTM slides system (Schleicher & Schuell, Dassel, Germany), which incorporates a patch of polymer on the surface of a glass slide.

In general, antigens on the array should be discrete, in that signals from that antigen can be distinguished from signals of neighboring antigens, either by the naked eye (macroarrays) or by scanning or reading by a piece of equipment or with the assistance of a microscope (microarrays).

Macro-arrays are often arrayed on polymer membranes, either supported or not, and can be of any size, but typically will be greater than a square centimeter. Other examples of macroarray substrates include glass, fiber, plastic and metal. Macroarrays are generally used when the number of antigens in the panel is relatively small, on the order of tens to hundreds of antigens, however macroarrays with a larger number of array elements can be used on large substrates. Spot arrangement on the macroarray is such that individual spots can be distinguished from each other when the binding is analyzed, typically, the diameter of the spot is about equal to the spacing between individual dots.

Sample spots on macroarrays are of a size large enough to permit their detection without the assistance of a microscope or other sophisticated enlargement equipment. Thus, spots may be as small as about 0.1 mm across, with a separation of about the same distance, and can be larger. Larger spots on macroarrays, for example, may be about 0.5, 1, 2, 3, 5, 7, or 10 mm across. Even larger spots may be larger than 10 mm (1 cm) across, in certain specific embodiments. The array size will in general be correlated the size of the spots applied to the array, in that larger spots will usually be found on larger arrays, while smaller spots may be found on smaller arrays. This correlation is not necessary to the invention, though.

In microarrays, a common feature is the small size of the antigen array, for example on the order of a squared centimeter or less. A squared centimeter (1 cm by 1 cm) is large enough to contain over 2,500 individual antigen spots, if each spot has a diameter of 0.1 mm and spots are separated by 0.1 mm from each other. A two-fold reduction in spot diameter and separation can allow for 10,000 such spots in the same array, and an additional halving of these dimensions would allow for 40,000 spots. Using microfabrication technologies, such as photolithography, pioneered by the computer industry, spot sizes of less than 0.01 mm are feasible, potentially providing for over a quarter of a million different target sites. The power of microarray format resides not only in the number of different antigens that can be probed simultaneously, but also in how little protein is needed for the spot.

The amount of antigen that is applied to each address of an array will be largely dependent on the array format used. For instance, microarrays will generally have less antigen applied at each address than will macroarrays. By way of example, individual antigens (in this case, peptides and polypeptides) on a macroarray can be applied in the amount of about 1 pmol or greater, for instance about 3 pmol, about 5 pmol, about 7.5 pmol, about 10 pmol, about 15 pmol or more. In contrast, samples applied to individual spots on a microarray will usually be less than 1 pmol in each spot, for instance, about 8 pmol, about 0.5 pmol, about 0.3 pmol, about 0.1 pmol, about 0.05 pmol or less.

In addition, the surface area of antigen application for each “spot” will influence how much antigen is immobilized on the array surface. Thus, a larger spot (having a greater surface area) will generally accept or require a greater amount of target molecule than a smaller sample spot (having a smaller surface area).

The antigen itself (e.g., the length of the peptide or polypeptide, its primary and secondary structure, its binding characteristics in relation to the array substrate, etc.) will influence how much of each antigen is applied to an array. Optimal amounts of antigen for application to an array of the invention can be easily determined, for instance by applying varying amounts of the antigen to an array surface and probing the array with an antibody known to interact with that antigen. In this manner, it is possible for one of ordinary skill in the art to empirically determine of range of antigen amounts that produce reproducible and interpretable results.

Another way to describe an array is its density—the number of antigens in a certain specified surface area. For macroarrays, array density will usually be between about one antigen per squared decimeter (or one antigen address in a 10 cm by 10 cm region of the array substrate) to about 50 antigens per squared centimeter (50 targets within a 1 cm by 1 cm region of the substrate). For microarrays, array density will usually be one target per squared centimeter or more, for instance about 50, about 100, about 200, about 300, about 400, about 500, about 1000, about 1500, about 2,500, about 5,000, about 10,000, about 50,000, about 100,000 or more targets per squared centimeter.

Antigens on the array may be made of oligopeptides, polypeptides, proteins, or fragments of these molecules. Oligopeptides, containing between about 8 and about 50 linked amino acids, can be synthesized readily by chemical methods. Photolithographic techniques allow the synthesis of hundreds of thousands of different types of oligopeptides to be separated into individual spots on a single chip, in a process referred to as in situ synthesis, as has been done with oligonucleotide arrays.

Longer polypeptides or proteins, on the other hand, contain up to several thousand amino acid residues, and are not as easily synthesized through in vitro chemical methods. Instead, polypeptides and proteins for use in antigen arrays are usually expressed using one of several well known cellular expression systems, including those described above. Alternatively, proteins can be isolated from their native environment, for instance from tissue samples or cell cultures, or from expression chambers in the case of engineered expressed polypeptides. After extraction and appropriate purification, the polypeptide can be deposited onto the array using any of a variety of techniques.

In the methods disclosed in this applications, antigens can be delivered to the substrate of the array by various different mechanisms. One is by flowing within a channel defined on predefined regions of the array substrate. Typical “flow channel” application methods for applying polypeptides to arrays are represented by dot-blot or slot-blot systems (see, e.g., U.S. Pat. Nos. 4,427,415 and 5,283,039). One alternative method for applying the antigens to the array substrate is “spotting” the antigens on predefined regions (each corresponding to an array address). In a spotting technique, the target molecules are delivered by directly depositing (rather than flowing) relatively small quantities of them in selected regions. For instance, a dispenser can move from address to address, depositing only as much antigen as necessary at each stop. Typical dispensers include an ink-jet printer or a micropipette to deliver the antigen in solution to the substrate and a robotic system to control the position of the micropipette with respect to the substrate. In other embodiments, the dispenser may include a series of tubes, a manifold, an array of pipettes, or the like so that the antigens can be delivered to the reaction regions simultaneously.

In a preferred embodiment, the antigens are deposited on the array substrate in such a way that they are substantially irreversibly bound to the array. For example, a target may be bound such that no more than 30% of the polypeptide on the array at the end of the binding process can be washed off using buffers (e.g., low or high salt buffers or stripping buffers). In other embodiments, no more than 25%, no more than 20%, no more than 15%, no more than 10%, no more than 5%, or no more than 3% of the antigen on the array at the end of the binding process can be washed off.

Depending on the array substrate used, the substrate alone may substantially irreversibly bind the antigen without further linking being necessary (e.g., nitrocellulose and PVDF membranes). In other instances, a linking or binding process must be performed to ensure binding of the antigens. Examples of linking processes are known to those of skill in the art, as are the substrates that require such a linking process in order to bind polypeptide molecules. The antigen polypeptides optionally may be attached to the array substrate through linker molecules.

In certain embodiments, the regions of the array surface that do not contain any antigens are blocked in order to prevent or inhibit binding of the antibody molecules directly to the array surface.

It is beneficial in certain embodiments to apply a known amount of each antigen to the array. For example, where the diagnostic test antigens are applied, it may be useful to have a known amount of the antigen. Moreover, in some modes, several doses of the known test antigens may be useful to quantitate antibody titer levels in the patient sample. In particular embodiments, an essentially equal amount of each antigen is applied to each spot. Quantification and equivalent application of the antigen permits comparison of antibody binding affinity between the different antigens. Measurements of the amount of specific proteins may be carried out through many techniques well known in the art.

Arraying pooled antigens spotted on the array is also a powerful tool in hi-throughput technologies for increasing, the information that is yielded each time the array is assayed. Methods for analyzing signals from arrays containing pooled samples have been described, for instance in U.S. Pat. No. 5,744,305, throughput incorporated herein by reference in its entirety.

1. A developer reagent for staining immobilized protein/human IgE antibody/conjugate (anti-human-IgE-Alkaline Phosphatase) complexes, comprising AP Buffer (0.1M Tris pH 9.5, 0.1M NaCl, 0.05M MgCl2), Nitro-Blue Tetrazolium Chloride (NBT), and 5-Bromo-4-Chloro-3′-Indolylphosphate p-Toluidine salt (BCIP).

2. A method for increasing signal to noise ratio in protein-display micro array chips, said method comprising washing said chips with a non-phosphate containing buffer.

3. The method as recited in claim 2, wherein said non-phosphate containing buffer is a 1× Tris Borate Saline buffer (composition: 8 g of NaCl, 0.2 g of KCl, and 3 g of Tris base in 800 ml of distilled water).

4. The method as recited in claim 2, wherein said non-phosphate containing buffer will increase signal to noise ratio in protein-display micro array chips in comparison to washing in phosphate buffers by not introducing exogenous phosphate groups into the micro array chips.

5. The method as recited in claim 4, wherein exogenous phosphate groups introduced into the system will create a lower signal to noise ratio.

6. The method recited in claim 5, wherein the lower signal to noise ratio is the result of Diformazan, which is a product of the reaction between Nitro-Blue Tetrazolium Chloride (NBT), and 5-Bromo-4-Chloro-3′-Indolylphosphate p-Toluidine salt (BCIP).

7. The method recited in claim 6, wherein Diformazan will form insoluble purple precipitate at sites of alkaline phosphatase activity.

8. The method recited in claim 5, wherein potential “competitive” reaction is a competition for Diformazan between exogenous phosphate groups and the phosphate group present in BCIP.
